(19) United States
(12) Patent Application Publication
(10) Pub. No.: US 2024/0293294 A1
(43) Pub. Date: Sep. 5, 2024

(54) SPHERULITE COMPOSITIONS

(71) Applicant: Southern Chemical & Textiles, Inc., Dalton, GA (US)

(72) Inventors: Lauren HOLSINGER, Monmouth Junction, NJ (US); James GRIFFIN, Monmouth Junction, NJ (US)

(73) Assignee: Southern Chemical & Textiles, Inc., Dalton, GA (US)

(21) Appl. No.: 18/275,718

(22) PCT Filed: Feb. 2, 2022
(86) PCT No.: PCT/US2022/014837

§ 371 Date: Aug. 3, 2023

Related U.S. Application Data
(60) Provisional application No. 63/149,884, filed on Feb. 16, 2021.

(51) Int. Cl. A61K 8/02; A61Q 19/10

(52) U.S. Cl. CPC A61K 8/025; A61Q 19/10; A61K 2800/30

(57) ABSTRACT

A spherulite concentrate combining water, a linear alkyl sulfate surfactant, and specific amphoteric surfactants. The concentrate can be used in a skin care composition, where the skin care composition contains the concentrate in an amount between, e.g., 70% and 99% by weight of the composition, cosmetically acceptable oils, and a pH adjuster. The skin care compositions may be present in packaging, which may contain one or more skin care compositions described herein.

### CROSS-REFERENCE TO RELATED APPLICATIONS

This application claims priority to U.S. Provisional Application No. 63/149,884, filed Feb. 16, 2021, the entirety of which is incorporated by reference herein.

### FIELD OF THE INVENTION

This invention relates to surfactant compositions, more particularly to structured surfactant compositions.

### BACKGROUND OF THE INVENTION

Structured surfactant compositions are liquid crystalline compositions that are useful in various applications, including, e.g., skin care formulations such as shampoos, body wash, hand soap, lotions, creams, conditioners, shaving products, and facial washes.

Surfactants in the structured surfactant compositions exist in the form of lamellar phases that are planar and/or in the form of spherulites. Commonly, the surfactant phase is present as spherulites, i.e., lamellar droplets, dispersed in the aqueous phase. Spherulites consist of an onion-like configuration of concentric bi-layers of surfactant molecules, between which is trapped water or electrolyte solution. Exclusively planar lamellar surfactant phases or exclusively spherulite lamellar surfactant phases or the combination of both forms can co-exist in the same composition. Structured surfactant compositions are typically pumpable, non-Newtonian compositions that have the capacity physically to suspend water insoluble particles by virtue of the presence of these lamellar surfactant phases.

Typically, electrolytes such as various salts are required in these structured surfactant compositions which increases zero shear viscosity (i.e., viscosity when sitting in bottle) helping to suspend particles but also increases critical shear rate (point at which product becomes shear thinning; higher critical shear rates mean product is more difficult to pour). Similarly, polymers, such as gums, are utilized to form or stabilize spherulites, but such polymers can negatively impact the rheology and/or aesthetics of the composition.

To avoid these tradeoff, new structured surfactant compositions that do not require the use of electrolytes and/or polymers is needed.

### BRIEF SUMMARY

According to a first aspect of the present disclosure, a spherulite concentrate may be provided, utilizing a specific combination of anionic and amphoteric surfactants.

Specifically, the concentrate uses between 5 and 25% by weight of an anionic alkyl sulfate surfactant, and more specifically, the concentrate utilizes sodium lauryl sulfate.

Further, the concentrate utilizes two amphoteric surfactants, a first amphoteric surfactant and a second amphoteric surfactant, each in amounts between 10 and 30% by weight. The first amphoteric surfactant may consist of disodium cocoamphodiacetate, sodium cocoamphoacetate, sodium lauroamphoacetate, and/or sodium cocoamphohydroxypropylsulfonate. The second amphoteric surfactant may consist of a hydroxysultaine, and in particular, cocamidopropyl hydroxysultaine. The first and second amphoteric surfactants are used at a ratio of between 1:1.1 and 1:1.5 of first amphoteric surfactant to second amphoteric surfactant.

In some embodiments, the concentrate may be free, or substantially free, of additional electrolytes, nonionic surfactants, polymers, and/or other anionic surfactants. In some embodiments, the concentrate may comprise at least 30% by weight of water. In some embodiments, the ratio of the total amount of amphoteric surfactants to the total amount of sodium lauryl sulfate may be between 1.5:1 and 2.5:1.

The first amphoteric surfactant may have one or more components. In some embodiments, the first amphoteric surfactant may consist of (i) disodium cocoamphodiacetate and (ii) sodium cocoamphoacetate, sodium lauroamphoacetate, and/or sodium cocoamphohydroxypropylsulfonate. In some embodiments, the first amphoteric surfactant may consist of only disodium cocoamphodiacetate, sodium cocoamphoacetate, sodium lauroamphoacetate, or sodium cocoamphohydroxypropylsulfonate.

In some embodiments, the spherulite concentrate may consist of sodium lauryl sulfate, the first amphoteric surfactant and the second amphoteric surfactant, and water. In some embodiments, the spherulite concentrate may consist of sodium lauryl sulfate, the first amphoteric surfactant and the second amphoteric surfactant, water, and a preservative.

According to a second aspect of the present disclosure, a skin care composition may be provided. The skin care composition may contain an embodiment of the spherulite concentrate described previously, and one or more additional components. In some embodiments, the skin care composition may comprise between 70% and 99% of the spherulite concentration, one or more cosmetically acceptable oils, and a pH adjuster.

In some embodiments, the skin care composition may contain a total of between 1% and 15% by weight of the cosmetically acceptable oils. In some embodiments, the skin care composition may include a preservative system, a fragrance, a colorant, an exfoliating agent, and/or an active agent.

According to a third aspect of the present disclosure, a skin care system may be provided. In some embodiments, the skin care system may include a hand-held container and a first skin care composition as described above.

In some embodiments, the skin care system may include a second skin care composition as described above, different from the first skin care composition, that is also positioned within the container, where a surface of the first skin care composition is in contact with a surface of the second skin care composition.

### DETAILED DESCRIPTION

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

As used herein, the term “between” used in a range is intended to include the recited endpoints. For example, a number “between x and y” can be x, y, or any value from x to y.

As used herein, the term “free” of a material means the material is not present in the composition in a detectable amount.

As used herein, the term “substantially free” of a material means the material is present in the composition in an amount less than or equal to 1% by weight of the composition.

The disclosed spherulite concentrates comprise or consist essentially of a combination of (i) water, (ii) a linear (i.e., non-branching) anionic alkyl sulfate surfactant, (iii) specific amphoteric surfactants, and (iv) optionally an additive such as a preservative, chelating agent, colorant, UV filter, fragrance, and/or antioxidant.

The spherulite concentrate contains water. Water will preferably be present in an amount greater than 30% of the total concentrate. In some embodiments, water is present in an amount of at least 25%, 30%, or 35% by weight of the concentrate, and no more than 75%, 65%, 55%, 45%, or 35% by weight of the concentrate, including all combinations of ranges and subranges thereof.

The spherulite concentrate utilizes at least one linear (i.e., non-branching) anionic alkyl sulfate surfactant, and preferably Sodium Lauryl Sulfate (CAS #151-21-3):

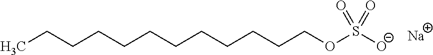 [SMILES: CCCCCCCCCCCCOS(=O)(=O)[O-] ; [Na+]]

In some embodiments, the total amount of alkyl sulfate surfactant present is at least 10%, 12%, or 15%, and no more than 30%, 28%, or 25% by weight of the concentrate, including all combinations of ranges and subranges thereof. Preferably, the alkyl sulfate surfactant is generally present in an amount of between 5 and 25% by weight of the concentrate.

The spherulite concentrate also utilizes amphoteric surfactants. The amphoteric surfactants comprise, and preferably consist of, two amphoteric surfactants, (B) and (C) as described below.

Each amphoteric surfactant is generally present in amounts that are between 10% and 30% by weight of the concentrate. In some embodiments, each amphoteric surfactant is present in an amount of at least 10%, 12%, 14%, 16%, or 18%, and no more than 30%, 28%, or 26% by weight of the concentrate, including all combinations of ranges and subranges thereof.

In preferred embodiments, the total amount of amphoteric surfactant present in the concentrate is between 35% and 55%, and more preferably between 40% and 50% by weight of the concentrate.

In some embodiments, the amphoteric surfactants are present in a greater amount than the amount of anionic surfactant. In some embodiments, the total amount of amphoteric surfactant is present in a ratio of between 1.5:1 and 2.5:1 of amphoteric surfactant to anionic surfactant.

The first amphoteric surfactant (B) may comprise or consist of an amphoacetate or an amphosulfonate, and preferably consists of one or more of (B1), (B2), (B3), and/or (B4):

(B1): Disodium Cocoamphodiacetate (DSCADA) (CAS #68650-39-5):

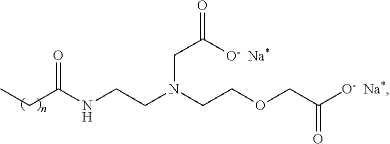

where typically n=6, 8, 10, 12, 14, 16;

(B2): Sodium Cocoamphoacetate (SCAA) (CAS #68334-21-4):

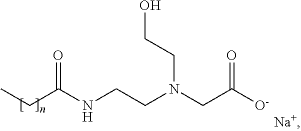

where typically n=6, 8, 10, 12, 14, 16;

(B3): Sodium Lauroamphoacetate (SLAA) (CAS #156028-14-7, 68647-44-9):

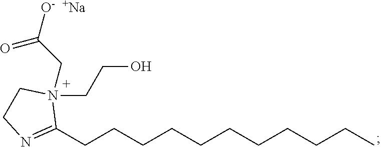

and/or

(B4): Sodium Cocoamphohydroxypropylsulfonate (SCAHPS) (CAS #68604-73-9):

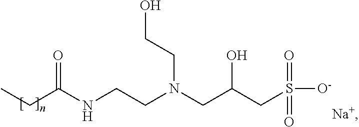

where typically n=6, 8, 10, 12, 14, 16.

In some embodiments, the first amphoteric surfactant (B) consists of (i) disodium cocoamphodiacetate (B1) and (ii) sodium cocoamphoacetate (B2), sodium lauroamphoacetate (B3), sodium cocoamphohydroxypropylsulfonate (B4), or a combination thereof.

In some embodiments, the first amphoteric surfactant consists of disodium cocoamphodiacetate, sodium cocoamphoacetate, sodium lauroamphoacetate, or sodium cocoamphohydroxypropylsulfonate. In some embodiments, the first amphoteric surfactant consists of disodium cocoamphodiacetate. In some embodiments, the first amphoteric surfactant consists of sodium cocoamphoacetate. In some embodiments, the first amphoteric surfactant consists of sodium lauroamphoacetate. In some embodiments, the first amphoteric surfactant consists of sodium cocoamphohydroxypropylsulfonate.

The amphoteric surfactant (C) comprises or consists of an hydroxysultaine, and preferably Cocamidopropyl Hydroxysultaine (CAS #68139-30-3):

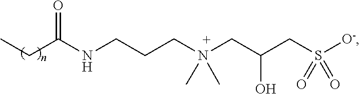

where typically n=6, 8, 10, 12, 14, 16.

In some embodiments, the first and second amphoteric surfactants are present in specific ratios of B:C of at least 1:1.1, 1:1.2, or 1:1.3, and no more than 1:1.6, 1:1.5, or 1:1.4, including all combinations of ranges and subranges thereof. Preferably, the ratio is between 1:1.1 and 1:1.5 of B:C.

The spherulite concentrate's surfactant system preferably consists of the linear (i.e., non-branching) anionic alkyl sulfate surfactant and the specific amphoteric surfactants. In some embodiments, the spherulite's surfactant system consists of (1) sodium lauryl sulfate in an amount of between 5% and 25% by weight, (2) a first amphoteric surfactant (B) (disodium cocoamphodiacetate, sodium cocoamphoacetate, sodium lauroamphoacetate, and/or sodium cocoamphohydroxypropylsulfonate) and (3) a second amphoteric surfactant (C) (cocamidopropyl hydroxysultaine), where each amphoteric surfactant present in an amount of between 10% and 30% by weight, the amphoteric surfactants provided at a ratio of A:B of between 1:1.1 and 1:1.5.

The spherulite concentrate may optionally contain additives. If present, such additives are preferably present in amounts less than 5%, 4%, 3%, or 2% by weight of the concentrate. More preferably, the formulations are substantially free of additives (that is, the total amount of additives is less than 1% by weight of the concentrate). Still more preferably, if present, the additives are present in an amount less than or equal to 0.5% by weight of the concentrate.

The additives may comprise or consist of a preservative, a chelating agent, a colorant, a UV filter, and/or an antioxidant.

The spherulite concentrate may optionally include a preservative system. Preferably, the formulations are substantially free of preservative (that is, the total preservative system utilized is less than 1% by weight of the concentrate.) Examples of materials that can be used as preservatives or in preservative systems include, e.g., alcohols, such as benzyl alcohol, phenoxyethanol, propylene, phenoxyethanol, phenethyl alcohol, 1,2-pentanediol, 1,2-hexanediol, 1,6-hexanediol, 1,2-octanediol, 1,2-decanediol, methylpropanediol and ethylhexylglycerol; and quaternary ammonium biocides (including those having efficacy against microbes) such as benzalkonium chloride, benzethonium chloride, didecyldimonium chloride, dioctyldimonium chloride; and combinations thereof.

The spherulite concentrates may optionally include a chelating agent, including, e.g., amino carboxylates, amino phosphonates, polyfunctionally-substituted aromatic chelating agents and mixtures therein. If utilized, these chelating agents will generally comprise 0.1% to less than 1% by weight of the concentrate.

The spherulite concentrates may optionally include a colorant, such as water-soluble or liposoluble dyestuff. Water-soluble dyes can include, e.g., synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophylline, methylene blue, anthocyanins (enocianin, black carrot, hibiscus and elder), caramel and riboflavin. Liposoluble dyes can include, e.g., synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

In some embodiments, the colorant may be a pigment. Non-limiting examples of suitable pigments include, but are not limited to, inorganic pigments, organic pigments, lakes, pearlescent pigments, iridescent or optically variable pigments, and mixtures thereof. A pigment should be understood to mean inorganic or organic, white or colored particles. Said pigments may optionally be surface treated within the scope of the present invention but are not limited to treatments such as silicones, perfluorinated compounds, lecithin, and amino acids.

Non-limiting examples of inorganic pigments include rutile or anatase titanium dioxide, coded in the Color Index under the reference CI 77,891; black, yellow, red, and brown iron oxides, coded under references CI 77, 499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof. Non-limiting examples of organic pigments and lakes include D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 28 Lake (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570), and mixtures thereof. Non-limiting examples of pearlescent pigments include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride, and mixtures thereof.

The spherulite concentrates may optionally include a fragrance. Such fragrances include the use of material that are intended to counteract, hide, or “mask” an offensive or undesirable natural odor of other materials in the composition, and preferably allow the concentrate to have a neutral odor.

The spherulite concentrates may optionally include one or more UV filters. UV filters include, e.g., para-Aminobenzoic acid derivatives such as PABA, ethyl PABA, or Ethylhexyl Dimethyl PABA; Dibenzoylmethane Derivatives such as Butyl Methoxydibenzoylmethane; Salicylic Derivatives such as Homosalate, or Ethylhexyl Salicylate, Cinnamic Derivatives such as Ethylhexyl Methoxycinnamate; β,β-Diphenylacrylate Derivatives, such as Octocrylene; Benzophenone Derivatives such as Benzophenone-1, -2, -3, -4, -5, or -6; and Benzylidene camphor derivatives such as 4-Methylbenzylidene camphor.

The spherulite concentrates may optionally include one or more antioxidants, such as vitamin C, vitamin A, vitamin E, resveratrol, niacinamide, or glutathione.

### Example 1 (Production of Concentrates)

To manufacture the concentrates, the materials and amounts as described in Tables 1A-1D, below, can be added to a mixing vessel, and heated to a temperature of 45° C. while being mixed. The concentrate can then be cooled to room temperature and transferred to an appropriate storage container.

TABLE 1A
(Concentrate Formulas)

| | Wt. % | | | |
|---|---|---|---|---|
| Ingredient | Ex. 1-1 | Ex. 1-2 | Ex. 1-3 | Ex. 1-4 |
| Water | 30-35% | 30-35% | 30-35% | 30-35% |
| Sodium Lauryl Sulfate | 20-25% | 20-25% | 20-25% | 20-25% |
| Disodium Cocoamphodiacetate | 18-20% | 9-10% | — | 16-20% |
| Sodium Cocoamphoacetate | — | 9-10% | 18-20% | 1-10% |
| Coamidopropyl Hydroxysultaine | 23-28% | 23-28% | 23-28% | 23-28% |
| Preservative System | 0.5-1% | 0.5-1% | 0.5-1% | 0.5-1% |

TABLE 1B
(Concentrate Formulas)

| | Wt. % | | | |
|---|---|---|---|---|
| Ingredient | Ex. 1-5 | Ex. 1-6 | Ex. 1-7 | Ex. 1-8 |
| Water | 30-35% | 30-35% | 30-35% | 30-35% |
| Sodium Lauryl Sulfate | 20-25% | 20-25% | 20-25% | 20-25% |
| Disodium Cocoamphodiacetate | 9-10% | — | 18-20% | 5-10% |
| Sodium Cocoamphoacetate | — | — | — | 5-10% |
| Sodium Lauroamphoacetate | 9-10% | 18-20% | 1-10% | 5-10% |
| Coamidopropyl Hydroxysultaine | 23-28% | 23-28% | 23-28% | 23-28% |
| Preservative System | 0.5-1% | 0.5-1% | 0.5-1% | 0.5-1% |

TABLE 1C
(Concentrate Formulas)

| | Wt. % | | | |
|---|---|---|---|---|
| | Ex. | Ex. | Ex. | Ex. |
| Ingredient | 1-9 | 1-10 | 1-11 | 1-12 |
| Water | 30-35% | 30-35% | 30-35% | 30-35% |
| Sodium Lauryl Sulfate | 20-25% | 20-25% | 20-25% | 20-25% |
| Disodium Cocoamphodiacetate | 9-10% | — | 18-20% | 3-7% |
| Sodium Cocoamphoacetate | — | — | — | 3-7% |
| Sodium Lauroamphoacetate | — | — | — | 3-7% |
| Sodium Cocoamphohydroxy- | 9-10% | 18-20% | 1-10% | 3-7% |
| propylsulfonate | | | | |
| Coamidopropyl Hydroxysultaine | 23-28% | 23-28% | 23-28% | 23-28% |
| Preservative System | 0.5-1% | 0.5-1% | 0.5-1% | 0.5-1% |

TABLE 1D
(Concentrate Formulas)

| | Wt. % | | | |
|---|---|---|---|---|
| | Ex. | Ex. | Ex. | Ex. |
| Ingredient | 1-13 | 1-14 | 1-13 | 1-14 |
| Water | 55-75% | 30-35% | 30-35% | 30-35% |
| Sodium Lauryl Sulfate | 5-10% | 20-25% | 20-25% | 20-25% |
| Disodium | 10-20% | 18-20% | 18-20% | 18-20% |
| Cocoamphodiacetate | | | | |
| Coamidopropyl | 10-20% | 23-28% | 23-28% | 23-28% |
| Hydroxysultaine | | | | |
| Preservative System | — | — | 0.5-0.75% | 0.5%-2% |
| Fragrance | — | — | 0.5-1% | 0.3-0.7% |
| Colorant | — | — | 0.1-1% | — |
| UV Filter | — | — | — | 0.5%-2% |
| Chelating Agent | — | — | — | 0.1-0.5% |
| Antioxidant | — | — | — | 0.1-0.5% |

Also disclosed are skin care compositions, which contain the concentrate above and additional components, such as cosmetically acceptable oils, pH adjusting agents, and preservatives.

The skin care compositions generally utilize between 1% and 99% by weight of the spherulite concentrate described previously. In some embodiments, the concentrate is present in an amount between 70% and 99% by weight of the skin care composition, and preferably between 80% and 90% by weight of the skin care composition.

In some embodiments, the skin care composition according to the invention may include at least one cosmetically acceptable oil. In some embodiments, the oils that may be present in the skin care composition may be chosen, alone or as a mixture, from:
(a) hydrocarbon-based oils, such as triglycerides consisting of fatty acid esters of glycerol, the fatty acids of which may have varied chain lengths from C₄ to C₂₄, these chains possibly being linear or branched, and saturated or unsaturated; these oils may be heptanoic or octanoic acid triglycerides, and may be grapeseed oil, alfalfa oil, apricot oil, avocado oil, baobab oil, barley oil, blackcurrant oil, candlenut oil, canola oil, castor oil, coconut oil, corn oil, cottonseed oil, evening primrose oil, hazelnut oil, jojoba oil, macadamia oil, marrow oil, millet oil, musk rose oil, olive oil, palm oil, passionflower oil, poppyseed oil, pumpkin oil, quinoa oil, rapeseed oil, rye oil, safflower oil, sesame oil, sesame seed oil, shea butter, shea oil, soybean oil, sunflower oil, sweet almond oil, wheatgerm oil, or wintergreen oil; or caprylic/capric acid triglycerides, for instance those sold by the company Stearineries Dubois or those sold under the names Miglyol 810®, 812® and 818® by the company Dynamit Nobel;
(b) ethers containing from 10 to 40 carbon atoms;
(c) linear or branched hydrocarbons of mineral or synthetic origin, such as petroleum jelly (petrolatum), polydecenes, hydrogenated polyisobutene such as Parleam®, squalane and liquid paraffins, and mixtures thereof;
(d) esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂>10 carbon atoms, for instance dicaprylyl carbonate, purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, or alcohol or polyalcohol heptanoates, octanoates, decanoates or ricinoleates, for instance propylene glycol dioctanoate; hydroxylated esters, for instance isostearyl lactate, diisostearyl malate or 2-octyldodecyl lactate; polyol esters and pentaerythritol esters;
(e) fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol, and
(f) higher fatty acids such as oleic acid, linoleic acid or linolenic acid, and mixtures thereof.

The cosmetically acceptable oils may include plant extracts, including cannabinoid oils such as cannabidiol (CBD) oil.

The cosmetically acceptable oils that may be used in the composition according to the invention may be non-volatile polydimethylsiloxanes (PDMS), polydimethylsiloxanes comprising alkyl or alkoxy groups, which are pendent and/or at the end of a silicone chain, these groups each contain from 2 to 24 carbon atoms, phenyl silicones, for instance phenyl trimethicones, phenyl dimethicones, phenyltrimethyl-siloxydiphenylsiloxanes, diphenyl dimethicones and diphenyl-methyldiphenyltrisiloxanes, and mixtures thereof.

In some embodiments, the composition according to the present invention includes a combination of synthetic esters and natural oils.

In some embodiments, the cosmetically acceptable oil is present in the composition in an amount of from about 1% to about 30% by weight, preferably from about 5% to about 25% by weight, and more preferably from about 5% to about 15% by weight, based on the total weight of the composition.

The skin care composition generally utilizes one or more pH adjusting agents, to adjust the pH to below 6. pH adjusting agents include, e.g., lactic acid, citric acid, sodium citrate, succinic acid, and phosphoric acid. In some embodiments, the pH adjusting agent or agents are present in a total amount of less than 2% by weight of the active pH adjusting agent. In some embodiments, the active pH adjusting agent or agents are present in a total amount of less than 1% by weight (i.e., be “substantially free” of pH adjusting agent). For example, if Citric Acid (50%) is used as the sole pH adjusting agent in an amount of 1.5% by weight, the total amount of active pH adjusting agent would be 0.75% by weight (1.5%×50% active).

The skin care composition may also include a preservative or preservative system. The preservative system may include additional preservatives beyond whatever preservatives may be used in the concentrate. Preferably, the skin care compositions formulations are substantially free of preservative (that is, the total preservative system utilized is less than 1% by weight of the composition.) Examples of materials that can be used as preservatives or in preservative systems include, e.g., alcohols, such as benzyl alcohol, phenoxyethanol, propylene, phenoxyethanol, phenethyl alcohol, 1,2-pentanediol, 1,2-hexanediol, 1,6-hexanediol, 1,2-octanediol, 1,2-decanediol, methylpropanediol and ethylhexylglycerol; and quaternary ammonium biocides (including those having efficacy against microbes) such as benzalkonium chloride, benzethonium chloride, didecyldimonium chloride, dioctyldimonium chloride; and combinations thereof.

The skin care composition may also comprise an exfoliating agent, typically utilized in compositions to enable scrubbing of the skin. In some embodiments, the exfoliating particles may be exfoliating or scrubbing particles of mineral, vegetable, or organic origin. In some embodiments, the exfoliating agent may include polyethylene beads or powder, such as those sold under the name Microthene MN 727 or Microthene MN 710-20 by Equistar or such as the powders sold under the name Gotalene 120 Colorless 2 by Dupont; nylon particles, such as those sold by Arkema under the name Orgasol 2002 Exd Nat Cos; poly(vinyl chloride) powder; pumice (INCI name) such as pumice 3/B from Eyraud; ground fruit kernel shells, such as ground materials derived from apricot kernels or walnut shells; sawdust, wood flour or cork flour; glass beads; alumina (aluminum oxide) (INCI name: Alumina), such as the product sold under the name Dermagrain 900 by Marketech International; sugar crystals; beads which melt during application on the skin, such as, for example, spheres based on mannitol and cellulose such as those sold under the Unisphere names by Induchem, agar-based capsules such as those sold under the Primasponge names by Cognis; spheres based on jojoba or jojoba esters such as those sold under the Florasphères names by Floratech; and/or their mixtures. In some embodiments, the skin care compositions comprise up to 5% by weight of natural jojoba beads. In some embodiments, the exfoliating agent is present in a total amount of less than 10% by weight. In some embodiments, the exfoliating agent is present in a total amount of less than 5% by weight. In some embodiments, the exfoliating agent is present in a total amount of less than 4% by weight. In some embodiments, the exfoliating agent is present in a total amount of less than 3% by weight. In some embodiments, the exfoliating agent is present in a total amount of less than 2% by weight. In some embodiments, the exfoliating agent is present in a total amount of less than 1% by weight.

Other additives, including those discussed in reference to the concentrate, may also be incorporated into the skin care composition, such as a preservative, a chelating agent, a colorant, a UV filter, and/or an antioxidant. Thus, the skin care composition may optionally contain additives in amounts less than 5%, 4%, 3%, or 2% by weight of the skin care composition. In some embodiments, the skin care composition is substantially free of additives (that is, the total amount of additives is less than 1% by weight of the skin care composition). In some embodiments, the additives are present in an amount less than or equal to 0.5% by weight of the skin care composition. In some embodiments, the skin care composition is free of additives.

### Example 2 (Skin Care Compositions)

To manufacture the skin care composition, the materials and amounts as described in Table 2, below, can be produced using one of two approaches.

If heating is not required (e.g., if all materials are liquid at room temperature, or are soluble in the composition at room temperature), then all materials can be added to a mixing vessel and mixed at room temperature until homogenous. The composition can then be transferred to an appropriate storage container.

If heat is required (e.g., if an oil with a melting point above room temperature is utilized), then the compositions can be produced by heating the spherulite concentrate and those materials that require heating to above the melting point, mixing until homogenous, cooling to room temperature, adding the remaining materials, and mixing until homogenous. The composition can then be transferred to an appropriate storage container.

TABLE 2
(Skin Care Composition Formula)

| | Wt % | | | | |
|---|---|---|---|---|---|
| Ingredient | Ex. 2-1 | Ex. 2-2 | Ex. 2-3 | Ex. 2-4 | Ex. 2-5 |
| Spherulite Concentrate | 85-90% | 80-90% | 85-90% | 90-95% | 65-80% |
| Cosmetic Oil(s) | 8-13% | 8-18% | 8-13% | 2-8% | 20-30% |
| pH Adjuster | 1-2% | 1-2% | 1-2% | 1-2% | 1-2% |
| (e.g., Citric Acid, 50%) | | | | | |
| Preservative System | 0.4-0.7% | 0.4-0.7% | 0.4-0.7% | 0.4-0.7% | 0.4-0.7% |
| Fragrance | — | 0.4-1% | — | — | 0.4-1% |
| Colorant | — | 0.2-1% | — | — | — |
| Exfoliating Agent | — | — | 0.5-2% | 0.5-2% | 0.5-2% |

### Example 3 (Skin Care Compositions)

Various skin care compositions using different combinations of cosmetic oils at 10% by weight of the composition were manufactured according to the method described for Example 2. Each composition comprised a spherulite concentrate of, e.g., Ex. 1-1, and either canola oil, CBD oil, petrolatum, baobab oil, avocado oil, sesame oil, or rapeseed oil.

### Example 4 (Exfoliating Body Wash)

Various skin care compositions using different concentrates at different levels of exfoliating agents, using the manufacturing method described for Example 2. Each composition comprised a spherulite concentrate of, e.g., Ex. 1-14, a cosmetically acceptable oil at 8-13% (such as grapeseed oil), a 0.5% of a preservative system, and between 0.5 and 5% by weight of an exfoliating agent (such as jojoba beads).

It should be recognized that any additional structuring or stabilizing agents, beyond the alkyl sulfate/amphoteric surfactant system described above, are not required in the concentrate or in the final product compositions. In preferred embodiments, the concentrates and compositions are free, or substantially free, of these additional structuring or stabilizing agents.

Such structuring or stabilizing agents that are not required include, e.g., non-ionic surfactants, additional electrolytes, polymers, and/or other anionic surfactants.

For example, nonionic surfactants are not required to form the spherulite. Thus, in some embodiments, the composition is free, or substantially free, of nonionic surfactants.

Nonionic surfactants include, but are not limited to amine oxides, fatty alcohols, alkoxylated alcohols, fatty acids, fatty acid esters, and alkanolamides. Such amine oxides comprise, (C₁₀-C₂₄) saturated or unsaturated branched or straight chain alkyl dimethyl oxides or alkyl amidopropyl amine oxides, such as for example, lauramine oxide, cocamine oxide, stearamine oxide, stearamidopropylamine oxide, palmitamidopropylamine oxide, decylamine oxide as well as mixtures thereof. Such fatty alcohols include, for example, (C₁₀-C₂₄) saturated or unsaturated branched or straight chain alcohols, more typically (C₁₀-C₂₀) saturated or unsaturated branched or straight chain alcohols, such as for example, decyl alcohol, lauryl alcohol, myristyl alcohol, cetyl alcohol, stearyl alcohol, oleyl alcohol, linoleyl alcohol and linolenyl alcohol, and mixtures thereof. Such alkoxylated alcohols include alkoxylated, typically ethoxylated, derivatives of (C₁₀-C₂₄) saturated or unsaturated branched or straight chain alcohols, more typically (C₁₀-C₂₀) saturated or unsaturated branched or straight chain alcohols, which may include, on average, from 1 to 22 alkoxyl units per molecule of alkoxylated alcohol, such as, for example, ethoxylated lauryl alcohol having an average of 5 ethylene oxide units per molecule. Mixtures of these alkoxylated alcohols may be used. Such fatty acids include (C₁₀-C₂₄) saturated or unsaturated carboxylic acids, more typically (C₁₀-C₂₂) saturated or unsaturated carboxylic acids, such as, for example, lauric acid, oleic acid, stearic acid, myristic acid, cetearic acid, isostearic acid, linoleic acid, linolenic acid, ricinoleic acid, elaidic acid, arichidonic acid, myristoleic acid, and palmitoleic acid, as well as neutralized versions thereof. Such fatty acid esters include esters of (C₁₀-C₂₄) saturated or unsaturated carboxylic acids, more typically (C₁₀-C₂₂) saturated or unsaturated carboxylic acids, for example, propylene glycol isostearate, propylene glycol oleate, glyceryl isostearate, and glyceryl oleate, and mixtures thereof. Such alkanolamides include aliphatic acid alkanolamides, such as cocamide MEA (coco monoethanolamide) and cocamide MIPA (coco monoisopropanolamide), as well as alkoxylated alkanolamides, and mixtures thereof.

Further, additional electrolytes are not required to form the spherulite. That is, while some amphoteric surfactants may contain or form electrolytes in solution, a skilled artisan will not be required to add additional electrolytes to form the spherulite. Thus, in some embodiments, the composition is free, or substantially free of additional electrolytes. Electrolytes are known compounds and include salts of multivalent anions, such as potassium pyrophosphate, potassium tripolyphosphate, and sodium or potassium citrate, salts of multivalent cations, including alkaline earth metal salts such as calcium chloride and calcium bromide, as well as zinc halides, barium chloride and calcium nitrate, salts of monovalent cations with monovalent anions, including alkali metal or ammonium halides, such as potassium chloride, sodium chloride, potassium iodide, sodium bromide, and ammonium bromide, alkali metal or ammonium nitrates, and polyelectrolytes, such as uncapped polyacrylates, polymaleates, or polycarboxylates, lignin sulfonates or naphthalene sulfonate formaldehyde copolymers.

For example, polymers are not required to form the spherulite or stabilize the spherulite. Thus, in some embodiments, the composition is free, or substantially free, of polymers. Polymers that are used in this capacity include, e.g., hydroxypropyl guar, xanthan gum, and (C₁₀-C₃₀) alkyl acrylate cross-polymer, and various polymeric quaternary compounds, including Polyquaternium-1, Polyquaternium-4, Polyquaternium-6, Polyquaternium-7, Polyquaternium-10, Polyquaternium-11, Polyquaternium-15, Polyquarternium-16, Polyquaternium-22, Polyquaternium-24, Polyquaternium-28, Polyquaternium-29, Polyquaternium-32, Polyquaternium-33, Polyquaternium-35, Polyquaternium-37, Polyquaternium-39, Polyquaternium-44, Polyquaternium-46, Polyquaternium-47, Polyquaternium-52, Polyquaternium-53, Polyquarternium-55, Polyquaternium-59, Polyquaternium-61, Polyquaternium-64, Polyquaternium-65, Polyquaternium-67, Polyquaternium-69, Polyquaternium-70, Polyquaternium-71, Polyquaternium-72, Polyquaternium-73, Polyquaternium-74, Polyquaternium-76, Polyquaternium-77, Polyquaternium-78, Polyquaternium-79, Polyquaternium-80, Polyquaternium-81, Polyquaternium-82, Polyquaternium-84, Polyquaternium-85, and Polyquaternium-87; and mixtures thereof.

Other structuring agents, including, e.g., other anionic surfactants are not required. Thus, the compositions are preferably free, or substantially free, of other anionic surfactants. Such other anionic surfactants can include, e.g., linear alkylbenzene sulfonates, alpha olefin sulfonates, paraffin sulfonates, alkyl ester sulfonates, alkyl sulfonates, alkyl alkoxy carboxylates, monoalkyl phosphates, dialkyl phosphates, sarcosinates, isethionates, and taurates. Specifically, such other anionic surfactants can include, ammonium lauryl sulfate, ammonium laureth sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium trideceth sulfate, sodium tridecyl sulfate, ammonium trideceth sulfate, ammonium tridecyl sulfate, sodium cocoyl isethionate, disodium laureth sulfosuccinate, sodium oleoyl n-methyl taurate, sodium laureth carboxylate, sodium trideceth carboxylate, sodium-monoalkyl phosphates, sodium dialkyl phosphates, sodium lauryl sarcosinate, lauroyl sarcosine, cocoyl sarcosinate, ammonium cocyl sulfate, sodium cocyl sulfate, potassium cocyl sulfate, monoethanolamine cocyl sulfate, sodium tridecyl benzene sulfonate, sodium dodecyl benzene sulfonate. Other anionic surfactants further include branched anionic surfactants, such as sodium trideceth sulfate, sodium tridecyl sulfate, ammonium trideceth sulfate, and ammonium tridecyl sulfate.

A third aspect of the present disclosure is drawn to a skin care system. The skin care system may include a container (such as a hand-held container) and a first skin care composition according to any of the embodiments described previously. In some embodiments, the container may be, e.g., a tube, bottle, or tub. In some embodiments, the container may include one opening. In some embodiments, the container may include a separate cap or covering configured to cover the one opening. In some embodiments, the container is configured to dispense the first skin care composition via squeezing the container or via a pump embedded in a cap coupled to the container. In some embodiments, the container may be a sealed or resealable flexible polymer or foil wrapping.

In some embodiments, the skin care composition may be in or on a substrate. For example, a non-woven material may be used as a substrate for a wipe, where the skin care composition is contained in or on the non-woven material.

In some embodiments, the skin care system may include one or more additional skin care compositions in the container. In some embodiments, the system may include a second skin care composition according to any of the embodiments described previously, positioned within the container, where a surface of the first skin care composition is in contact with a surface of the second skin care composition. The second skin care composition must be different in some way from the first skin care composition. In some embodiments, the second skin care composition contains one or more colorants that is/are not present in the first skin care composition. In some embodiments, the second skin care composition contains one or more cosmetically acceptable oils that is/are not present in the first skin care composition. In some embodiments, the plurality of skin care compositions are layered within the container. In some embodiments, the plurality of skin care compositions are swirled within the container (such as in a helical pattern).

Those skilled in the art will recognize or be able to ascertain using no more than routine experimentation many equivalents to the specific embodiments of the invention described herein. Such equivalents are intended to be encompassed by the following claims.

1. A spherulite concentrate, comprising:
sodium lauryl sulfate in an amount of between 5% and 25% by weight; and
a first amphoteric surfactant and a second amphoteric surfactant;
wherein the first amphoteric surfactant consists of disodium cocoamphodiacetate, sodium cocoamphoacetate, sodium lauroamphoacetate, sodium cocoamphohydroxypropylsulfonate, or a combination thereof;
wherein the second amphoteric surfactant consists of cocamidopropyl hydroxysultaine; and
wherein the first amphoteric surfactant and the second amphoteric surfactant are each present in an amount of 10%-30% by weight, and the ratio R1 of the total amount of the first amphoteric surfactant to the total amount of the second amphoteric surfactant is 1:1.1≥R1≥1:1.5.

2. The spherulite concentrate according to claim 1, wherein the spherulite concentrate is substantially free of additional electrolytes.

3. The spherulite concentrate according to claim 1, wherein the spherulite concentrate is substantially free of nonionic surfactants.

4. The spherulite concentrate according to claim 1, wherein the spherulite concentrate is substantially free of other anionic surfactants.

5. The spherulite concentrate according to claim 1, wherein the spherulite concentrate is substantially free of polymers.

6. The spherulite concentrate according to claim 1, wherein the spherulite concentrate comprises at least 30% water.

7. The spherulite concentrate according to claim 1, wherein the ratio R2 of the total amount of amphoteric surfactants to the total amount of sodium lauryl sulfate is 1.5:1≥R2≥2.5:1.

8. The spherulite concentrate according to claim 1, wherein the first amphoteric surfactant consists of:
disodium cocoamphodiacetate; and
sodium cocoamphoacetate, sodium lauroamphoacetate, sodium cocoamphohydroxypropylsulfonate, or a combination thereof.

9. The spherulite concentrate according to claim 8, wherein the first amphoteric surfactant consists of:
disodium cocoamphodiacetate; and
sodium cocoamphoacetate, sodium lauroamphoacetate, or sodium cocoamphohydroxypropylsulfonate.

10. The spherulite concentrate according to claim 1, wherein the first amphoteric surfactant consists of disodium cocoamphodiacetate, sodium cocoamphoacetate, sodium lauroamphoacetate, or sodium cocoamphohydroxypropylsulfonate.

11. The spherulite concentrate according to claim 1, wherein the spherulite concentration consists of:
sodium lauryl sulfate;
the first amphoteric surfactant and the second amphoteric surfactant;
water; and
a preservative.

12. A skin care composition, comprising:
spherulite concentrate according to claim 1, in an amount between 70% and 99% by weight of the skin care composition; and
one or more cosmetically acceptable oils; and
a pH adjuster.

13. The skin care composition according to claim 12, wherein the one or more cosmetically acceptable oils are present in an amount of between 1% and 15% by weight.

14. The skin care composition according to claim 12, further comprising a preservative system, a fragrance, a colorant, an exfoliating agent, an active agent, or a combination thereof.

15. A skin care system, comprising:
a container; and
a first skin care composition according to claim 9 positioned within the container.

16. The skin care system according to claim 15, further comprising a second skin care composition according to claim 9 positioned within the container, where a surface of the first skin care composition is in contact with a surface of the second skin care composition.

17. The skin care system according to claim 15, wherein the first skin care composition is in contact with a substrate, the substrate being positioned within the container.
